(19) United States
(12) Patent Application Publication
(10) Pub. No.: US 2020/0227147 A1
(43) Pub. Date: Jul. 16, 2020

(54) AUTOMATED GENERATION OF CODES

(71) Applicant: 3M INNOVATIVE PROPERTIES COMPANY, St. Paul, MN (US)

(72) Inventors: Nicholas J. Raddatz, St. Paul, MN (US); Dominick R. Rocco, Minneapolis, MN (US)

(21) Appl. No.: 16/738,549

(22) Filed: Jan. 9, 2020

Related U.S. Application Data
(60) Provisional application No. 62/790,836, filed on Jan. 10, 2019.

(51) Int. Cl. G16H 10/60; G06Q 50/22; G06N 20/00; G06F 40/20; G06N 3/04

(52) U.S. Cl. CPC G16H 10/60; G06Q 50/22; G06N 3/04; G06F 40/20; G06N 20/00

(57) ABSTRACT

A computer implemented method includes receiving text-based clinical documentation corresponding to a patient treated at a healthcare facility, converting the text-based clinical documentation to create a machine compatible converted input having multiple features, providing the converted input to a trained machine learning model that has been trained based on a training set of historical converted clinical documentation by the first entity, and receiving a prediction from the trained machine learning model, wherein the prediction corresponds to at least one of a predicted diagnostic related group (DRG) code or a set of predictions comprising a predicted principal diagnosis code for provision to a DRG calculator to determine the DRG code.

### BACKGROUND

Determination of the Diagnostic Related Group (DRG) corresponding to a medical encounter is an increasingly vital component in hospital prioritization and quality initiatives. DRG determination requires assignment of medical codes for principal diagnosis, secondary diagnoses, and procedures. Currently, the code assignment step requires significant human intervention, even when using the computer-assisted coding (CAC) tools in systems like 3M 360 Encompass.

### SUMMARY

A computer implemented method includes receiving text-based clinical documentation corresponding to a patient treated at a healthcare facility, converting the text-based clinical documentation to create a machine compatible converted input having multiple features, providing the converted input to a trained machine learning model that has been trained based on a training set of historical converted clinical documentation by the first entity, and receiving a prediction from the trained machine learning model, wherein the prediction corresponds to at least one of a predicted diagnostic related group (DRG) code or a set of predictions comprising a predicted principal diagnosis code for provision to a DRG calculator to determine the DRG code.

### BRIEF DESCRIPTION OF THE DRAWINGS

FIG. 1 is flowchart illustrating a machine implemented method of predicting codes based on clinical documentation according to an example embodiment.

FIG. 2 is a block flow diagram of a computer implemented method for generating a Diagnosis Related Group (DRG) code according to an example embodiment.

FIG. 3 is a block flow diagram of an alternative computer implemented method for generating a Diagnosis Related Group (DRG) code according to an example embodiment.

FIG. 4 is flowchart illustrating a machine implemented method of training a code predictor according to an example embodiment.

FIG. 5 is a block diagram of an example of an environment including a system for neural network training according to an example embodiment.

FIG. 6 is a block schematic diagram of a computer system to perform methods and algorithms according to example embodiments.

### DETAILED DESCRIPTION

In the following description, reference is made to the accompanying drawings that form a part hereof, and in which is shown by way of illustration specific embodiments which may be practiced. These embodiments are described in sufficient detail to enable those skilled in the art to practice the invention, and it is to be understood that other embodiments may be utilized and that structural, logical and electrical changes may be made without departing from the scope of the present invention. The following description of example embodiments is, therefore, not to be taken in a limited sense, and the scope of the present invention is defined by the appended claims.

The functions or algorithms described herein may be implemented in software in one embodiment. The software may consist of computer executable instructions stored on computer readable media or computer readable storage device such as one or more non-transitory memories or other type of hardware-based storage devices, either local or networked. Further, such functions correspond to modules, which may be software, hardware, firmware or any combination thereof multiple functions may be performed in one or more modules as desired, and the embodiments described are merely examples. The software may be executed on a digital signal processor, ASIC, microprocessor, or other type of processor operating on a computer system, such as a personal computer, server or other computer system, turning such computer system into a specifically programmed machine.

The functionality can be configured to perform an operation using, for instance, software, hardware, firmware, or the like. For example, the phrase “configured to” can refer to a logic circuit structure of a hardware element that is to implement the associated functionality. The phrase “configured to” can also refer to a logic circuit structure of a hardware element that is to implement the coding design of associated functionality of firmware or software. The term “module” refers to a structural element that can be implemented using any suitable hardware (e.g., a processor, among others), software (e.g., an application, among others), firmware, or any combination of hardware, software, and firmware. The term, “logic” encompasses any functionality for performing a task. For instance, each operation illustrated in the flowcharts corresponds to logic for performing that operation. An operation can be performed using, software, hardware, firmware, or the like. The terms, “component,” “system,” and the like may refer to computer-related entities, hardware, and software in execution, firmware, or combination thereof. A component may be a process running on a processor, an object, an executable, a program, a function, a subroutine, a computer, or a combination of software and hardware. The term, “processor,” may refer to a hardware component, such as a processing unit of a computer system.

Furthermore, the claimed subject matter may be implemented as a method, apparatus, or article of manufacture using standard programming and engineering techniques to produce software, firmware, hardware, or any combination thereof to control a computing device to implement the disclosed subject matter. The term, “article of manufacture,” as used herein is intended to encompass a computer program accessible from any computer-readable storage device or media. Computer-readable storage media can include, but are not limited to, magnetic storage devices, e.g., hard disk, floppy disk, magnetic strips, optical disk, compact disk (CD), digital versatile disk (DVD), smart cards, flash memory devices, among others. In contrast, computer-readable media, i.e., not storage media, may additionally include communication media such as transmission media for wireless signals and the like.

Diagnosis Related Group (DRG) code identification systems are a common tool used by healthcare payers and providers to classify treatments delivered to patients. By virtue of grouping encounters into categories, DRG code identification systems also allow providers to see expected metrics for each DRG, such as length of stay, cost of care, readmission rate, etc. Historically, DRG code identification systems have been used to set reimbursement levels for treatments and for submitting claims to prospective payment and value-based compensation schemes in health care.

It is becoming increasingly common, however, for hospitals to use automated DRG code identification systems as a tool for creating quality initiatives and prioritizing effort. Example DRG codes that might help with increasing quality include: Clinical Documentation Improvement—DRG 870 (sepsis) is commonly missing viral/bacterial specification, Case management and discharge planning—DRG 882 has 11 day mean length of stay, but patient has been in hospital for 15 days; and Quality initiatives—e.g. DRG 882 has high rates of readmission.

The trouble with DRG-based initiatives, however, is that hospital encounters must be coded to provide input for DRG determination. The DRG is determined for an inpatient encounter by a deterministic algorithm that takes the encounter principal diagnosis code, secondary codes, procedure codes, and patient demographic information as inputs. In practice, this means that a human medical coder must determine the principal diagnosis code for an encounter as well as codes for any relevant secondary diagnoses and procedures. As a result, hospitals cannot obtain a DRG for a patient until a human has determined the requisite codes. The dependence on a human coder introduces lag time into any quality or prioritization initiative based upon DRG values. In many cases, the coding work is not completed until the patient has left the hospital.

Various embodiments of the present inventive subject matter include a code predictor that predicts codes based on text-based clinical documentation. The codes may be a DRG code, or diagnosis and procedure codes for use by a DRG grouping algorithm to arrive at a DRG code.

The code predictor helps in assigning a DRG code prior to discharge by predicting inputs to the grouping algorithm (or a DRG itself) that normally would have been assigned by human review and coding. In other words, obtaining a DRG code normally involves a human sitting down to assign diagnosis and procedure codes, as well as to identify a principal diagnosis. The code predictor uses machine learning to predict values that a human might have assigned, without the human sitting down to do that job, but does so in a very different way than a human would do. The code predictor may be used to reduce or eliminate human involvement in DRG calculation by leveraging Machine Learning (ML) and Natural Language Processing (NLP) technology to automatically determine the DRG code/value or the medical codes as inputs for the DRG grouping algorithm. There are specific inputs to DRG grouping algorithm that are ideal for NLP extraction and ML estimation; namely medical codes corresponding to principal diagnosis, secondary diagnoses, and procedures. These inputs are complimented by other information that need not be predicted or estimated, such as age and gender.

The prediction of the inputs to DRG grouping algorithm may have value to users even in absence of DRG calculation. In other words, certain hospital roles and functions (e.g. prioritization initiatives) may wish to obtain a principal diagnosis code, for example, regardless of whether the DRG value is necessary for that role or function.

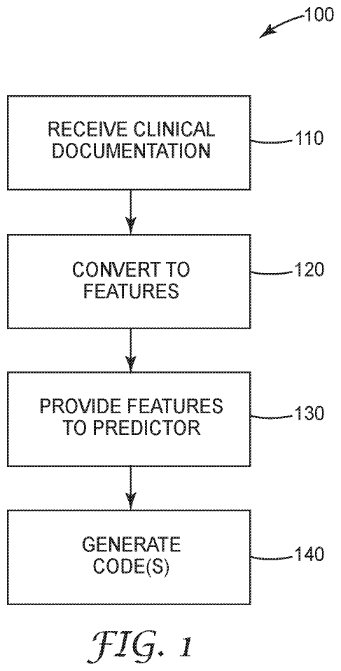

FIG. 1 is a flowchart illustrating a machine implemented method 100 of predicting codes based on clinical documentation. At operation 110, method 100 begins by receiving text-based clinical documentation corresponding to a patient treated at a healthcare facility.

At operation 120, the method continues by converting the text-based clinical documentation to create a machine compatible converted input having multiple features. Converting the text-based clinical documentation may include separating punctuation marks from text in the request and treating individual entities as tokens. Converting the text-based clinical documentation may be performed by a natural language processing machine and may include tokenizing the text-based clinical documentation to create tokens.

The converted input is provided at operation 130 to a trained machine learning model that has been trained based on a training set of historical converted clinical documentation by the first entity. The trained machine learning model may include a classification model such as a logistic regression model, support vector machine, decision tree, or nearest-neighbors algorithm. In some embodiments, the trained machine learning model comprises a recurrent or convolutional neural network. The training set may include patient demographics from a patient information database.

At operation 140, a prediction is received from the trained machine learning model. The prediction corresponds to at least one code. The at least one code may comprise a predicted diagnostic related group (DRG) code or a set of predictions including one or more of a predicted principal diagnosis code, a predicted secondary diagnosis code, and a predicted procedure code for provision to a DRG calculator to determine the DRG code. The set of predictions may include zero of more secondary procedure codes and zero or more predicted procedure codes for various different patient encounters.

In one embodiment, the machine learning model for predicting the code is trained on a training set that includes an associated DRG code corresponding to each treated patient in the historical converted clinical documentation such that the model is trained in a supervised manner.

In a further embodiment, the machine learning model for predicting set of predictions comprises is trained on the training set that includes an associated diagnosis or procedure code corresponding to each treated patient in the historical converted clinical documentation. The training set may include multiple secondary diagnosis codes and procedure codes for one or more treated patients in the historical converted clinical documentation. In this embodiment, the resulting diagnosis and prodcedure codes may be provided to a DRG grouping algorithm to determine a single corresponding DRG code.

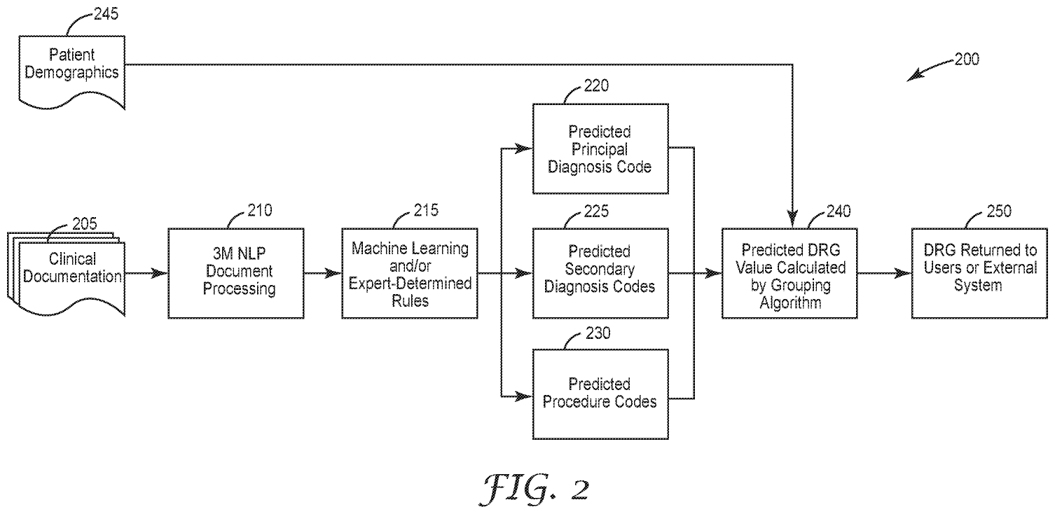

FIG. 2 is a block flow diagram illustrating components used in a system 200 to generate a DRG code from clinical documentation 205. The clinical documentation is provided to a natural language processing system 210 to convert the documentation into a machine compatible set of features. The features are provided to a code predictor 215. The code predictor 215 in some embodiments may be a trained machine learning model that has been trained in a supervised manner based on a training set of historical converted clinical documentation that includes associated medical diagnosis and procedure codes for each of multiple patient encounters.

An output of the code predictor 215 includes one or more diagnosis codes such as a predicted principal diagnosis code 220 and zero or more predicted secondary diagnosis codes 225. In addition, zero or more predicted procedure codes 230 may be included in the output. The codes are provided to a known DRG calculator 240 that may also receive patient demographics from a database 245. The DRG calculator 240 uses the received information to generate a single DRG code that may be returned via an output 250 to a user or further automated systems to generate requests for reimbursement and may also be used to enhance medical facility operations and improve patient care as well as economic performance of medical facilities.

The resulting DRG code, also referred to as a DRG value for a medical encounter is based on the clinical documentation 205 for that encounter, as well as the demographic information 245 that is received as discrete fields from an electronic health record (EHR) system. Existing NLP technology for system 210 may be used to extract information from the clinical documentation. The extracted information can be passed to the code predictor 215 which may comprise ML algorithms and/or a system of expert-determined rules. In the case where inputs to DRG algorithms are predicted, the ML algorithms and rules are used to select principal diagnosis codes 220, secondary diagnosis codes 225, and procedure codes 230. Those inputs are then passed
along with demographic information—to the DRG grouping algorithm 240 to calculate the DRG value and pass it along to an output 250.

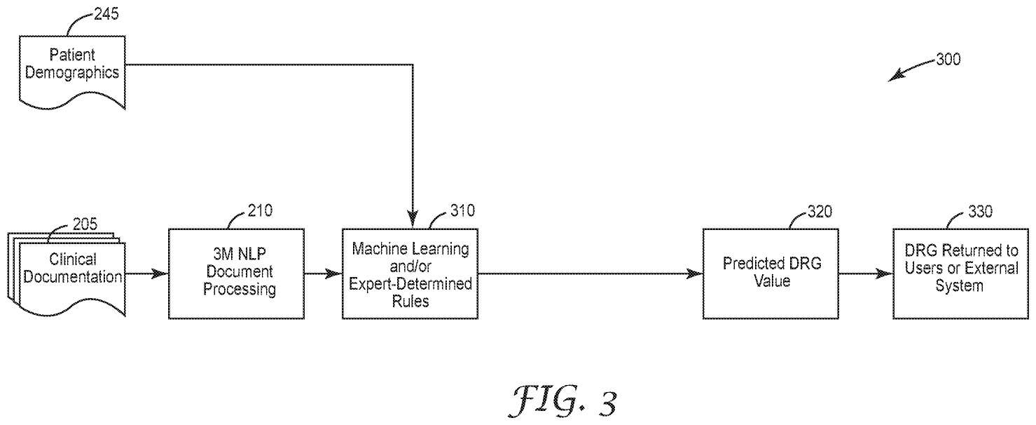

Alternatively, if DRG value is predicted directly as illustrated in an alternative system 300 in FIG. 3 where the references numbers are the same for like components. In system 300, a code predictor 310 receives the features from system 210, and utilizes ML algorithms and rules predict the DRG value 320 itself based on NLP generated features and demographic information, without passing any predicted values to a DRG calculation algorithm. If ML based, the code predictor 310 may be trained on the features of a training set having associated DRG codes to enable training in a supervised manner. The DRG value 320 is passed on via an output 330 to other systems and/or users.

An example of the clinical documentation that may be provided to the engine 210 to generate features used in both training the code predictors 215 and 310 is provided as follows:
Marvel General Hospital 01/20/2017
Attending Physician: Clark Kent, MD
Patient name: Wonder Woman

History of Present Illness
Patient is an adult female with a chief complaint of abdominal pain.
Patient reports a history of cigarette use, anxiety, and depression.
Patient reports pain in the upper-right abdomen region, is feeling indigestion and occasionally suffering from nausea and vomiting.

Diagnosis
An ultrasound exam was performed to identify gallstones with obstruction as likely cause for symptoms.

Treatment
Gall bladder was removed to eliminate issues caused by gall bladder.

Note that the raw clinical documentation shown above does not include the medical codes or the DRG code that is used for training such codes may be generated using prior methods, such as by human or DRG grouping code assist, and included in the training data.

Based on the above example clinical documentation, the engine 210 generates the following example feature set:

{

“diagnosis_codes”: [

| | |
|---|---|
| {“code”: “F41.9”, “description”: “Anxiety disorder, | unspecified”}, |
| {“code”: “R10.9”, “description”: “Unspecified abdominal | pain”}, |
| {“code”: “F17.210”, “description”: “Nicotine dependence, | cigarettes, |

uncomplicated”},

| | |
|---|---|
| {“code”: “R11.2”, “description”: “Nausea with vomiting, | unspecified”}, |

| | |
|---|---|
| {“code”: “F32.9”, “description”: “Major depressive | disorder, single episode, |

unspecified<”},

{“code”: “K80.01”, “description”: “Calculus of gallbladder with acute

cholecystitis, with obstruction”},

],
“procedure_codes”: [

| | |
|---|---|
| {“code”: “0FB44ZZ”, “description”: “Excision of | Gallbladder, |

Percutaneous Endoscopic Approach”},

| | |
|---|---|
| {“code”: “BH49ZZZ”, “description”: “Ultrasonography of | Abdominal |

Wall”}

]
“concepts:” [

{“id”: “x234”, “description”: “female patient”},
{“id”: “xs3591”, “description”: “abdominal pain”},
{“id”: “d3334”, “description”: “anxiety”},
{“id”: “a3234”, “description”: “depression”},
{“id”: “fd4546”, “description”: “indigestion”},
{“id”: “df453254”, “description”: “nausea”},
{“id”: “gf32353”, “description”: “gallstones”},
{“id”: “od245245”, “description”: “vomiting”}

]

}

Note that the features contain one or more diagnosis codes—F41.9; R10.9; F17.210; R11.2; F32.9; and K80.01 as well as two procedure codes—0FB44ZZ and BH49ZZZ. Note also that multiple concepts were extracted having different identifiers corresponding to female patient, abdominal pain, anxiety, depression, indigestion, nausea, gallstones, and vomiting. Each of these are features that are used in training the code predictors 215 and 310, along with corresponding codes.

Code predictor 215 will receive the features and provide the set of predicted diagnosis and procedure codes. As previously indicated, the set of predicted codes may include zero or more secondary diagnosis and procedure codes in addition to a predicted primary diagnosis code. An example output of code predictor 215 based on the example features above is as follows:

{
“principal_diagnosis”: {“code”: “K80.01”, “description”: “Calculus of gallbladder with acute cholecystitis, with obstruction” },

“DRG”: {“code”: “446”, “description”: “DISORDERS OF THE BILIARY

TRACT W/O CC/MCC”},

“diagnosis_codes”: [

{“code”: “F41.9”, “description”: “Anxiety disorder, unspecified”},
{“code”: “R10.9”, “description”: “Unspecified abdominal pain”},
{“code”: “F17.210”, “description”: “Nicotine dependence, cigarettes,

uncomplicated”},

{“code”: “R11.2”, “description”: “Nausea with vomiting, unspecified”},
{“code”: “F32.9”, “description”: “Major depressive disorder, single

episode, unspecified<”},

{“code”: “K80.01”, “description”: “Calculus of gallbladder with acute

cholecystitis, with obstruction”},

],
“procedure_codes”: [

{“code”: “0FB44ZZ”, “description”: “Excision of Gallbladder,

Percutaneous Endoscopic Approach”},

{“code”: “BH49ZZZ”, “description”: “Ultrasonography of Abdominal

Wall”}

]

}

The above output of the code predictor 215 includes a principal diagnosis code of K80.01 and multiple diagnosis codes, or secondary diagnosis codes listed in order of probability. The output may also include a DRG code prediction of 446—DISORDERS OF THE BILIARY TRACT W/O CC/MCC. Such an output may be obtained by training the code predictor to include diagnosis codes, prodedure codes, and DRG codes, in effect combining code predictors 215 and 310 into a single trained model using training data labeled with all the corresponding codes.

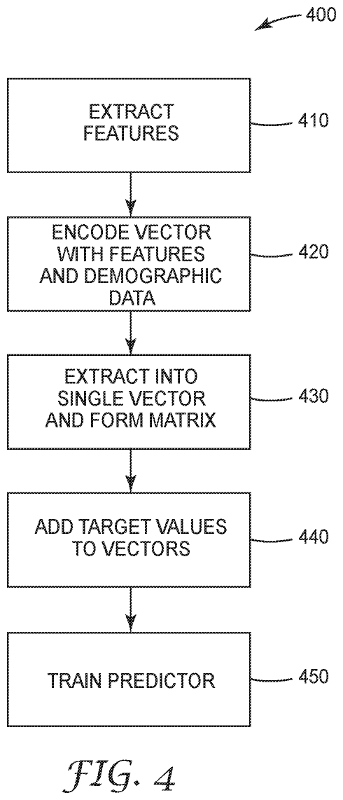

FIG. 4 is a flowchart illustrating a method 400 of training the code predictors. At operation 410, method 400 begins by extracting features (codes and/or concepts) from the clinical record training data by the NLP engine 210 and converting the features to a binary format using one-hot encoding where each possible code is represented by an element in a vector that may be zero or one, or also correspond to the number of identified occurrences within the document.

At operation 420, demographic information and other features are encoded into a feature vector as appropriate. For example, continuous or ordinal values are scaled to unit range. As another example, gender is one-hot encoded.

At operation 430, NLP and demographic features are concatenated into a single feature vector for each patient encounter and formed into a matrix containing many encounters. The training data may include hundreds to thousands of patient encounter medical records in various embodiments to obtain desired accuracy.

At operation 440, target values (principal diagnosis or DRG) are identified for each patient encounter are assembled into a vector with ordering corresponding to patient encounter feature matrix.

At operation 450, a machine learning algorithm (such as Logistic Regression, Support Vector Machine, Artificial Neural Network, Decision Tree, Boosted Decision Tree, Random Forest, k-Nearest Neighbors) is trained on the training data to predict target values (either principal diagnosis or DRG).

In one embodiment, an alternative deep-learning approach may be used to bypasses operations 410 and 430 in favor of using a deep learning algorithm with raw text medical records as inputs to a deep learning algorithm (such as Long short-term memory or convolutional neural network) to predict the target (principal diagnosis or DRG).

Artificial intelligence (AI) is a field concerned with developing decision making systems to perform cognitive tasks that have traditionally required a living actor, such as a person. Artificial neural networks (ANNs) are computational structures that are loosely modeled on biological neurons. Generally, ANNs encode information (e.g., data or decision making) via weighted connections (e.g., synapses) between nodes (e.g., neurons). Modern ANNs are foundational to many AI applications, such as automated perception (e.g., computer vision, speech recognition, contextual awareness, etc.), automated cognition (e.g., decision-making, logistics, routing, supply chain optimization, etc.), automated control (e.g., autonomous cars, drones, robots, etc.), among others.

Many ANNs are represented as matrices of weights that correspond to the modeled connections. ANNs operate by accepting data into a set of input neurons that often have many outgoing connections to other neurons. At each traversal between neurons, the corresponding weight modifies the input and is tested against a threshold at the destination neuron. If the weighted value exceeds the threshold, the value is again weighted, or transformed through a nonlinear function, and transmitted to another neuron further down the ANN graph—if the threshold is not exceeded then, generally, the value is not transmitted to a down-graph neuron and the synaptic connection remains inactive. The process of weighting and testing continues until an output neuron is reached; the pattern and values of the output neurons constituting the result of the ANN processing.

The correct operation of most ANNs relies on correct weights. However, ANN designers do not generally know which weights will work for a given application. Instead, a training process is used to arrive at appropriate weights. ANN designers typically choose a number of neuron layers or specific connections between layers including circular connection, but the ANN designer does not generally know which weights will work for a given application. Instead, a training process generally proceeds by selecting initial weights, which may be randomly selected. Training data is fed into the ANN and results are compared to an objective function that provides an indication of error. The error indication is a measure of how wrong the ANN's result was compared to an expected result. This error is then used to correct the weights. Over many iterations, the weights will collectively converge to encode the operational data into the ANN. This process may be called an optimization of the objective function (e.g., a cost or loss function), whereby the cost or loss is minimized.

A gradient descent technique is often used to perform the objective function optimization. A gradient (e.g., partial derivative) is computed with respect to layer parameters (e.g., aspects of the weight) to provide a direction, and possibly a degree, of correction, but does not result in a single correction to set the weight to a “correct” value. That is, via several iterations, the weight will move towards the “correct,” or operationally useful, value. In some implementations, the amount, or step size, of movement is fixed (e.g., the same from iteration to iteration). Small step sizes tend to take a long time to converge, whereas large step sizes may oscillate around the correct value or exhibit other undesirable behavior. Variable step sizes may be attempted to provide faster convergence without the downsides of large step sizes.

Backpropagation is a technique whereby training data is fed forward through the ANN—here “forward” means that the data starts at the input neurons and follows the directed graph of neuron connections until the output neurons are reached—and the objective function is applied backwards through the ANN to correct the synapse weights. At each step in the backpropagation process, the result of the previous step is used to correct a weight. Thus, the result of the output neuron correction is applied to a neuron that connects to the output neuron, and so forth until the input neurons are reached. Backpropagation has become a popular technique to train a variety of ANNs.

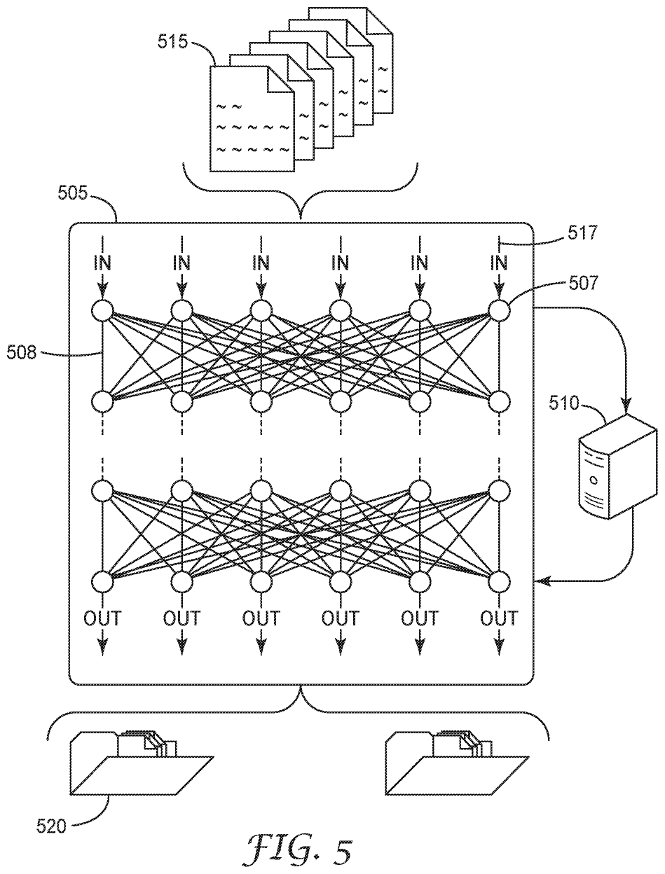

FIG. 5 is a block diagram of an example of an environment including a system for neural network training, according to an embodiment. The system includes an ANN 505 that is trained using a processing node 510. The processing node 510 may be a CPU, GPU, field programmable gate array (FPGA), digital signal processor (DSP), application specific integrated circuit (ASIC), or other processing circuitry. In an example, multiple processing nodes may be employed to train different layers of the ANN 505, or even different nodes 507 within layers. Thus, a set of processing nodes 510 is arranged to perform the training of the ANN 505.

The set of processing nodes 510 is arranged to receive a training set 515 for the ANN 505. The ANN 505 comprises a set of nodes 507 arranged in layers (illustrated as rows of nodes 507) and a set of inter-node weights 508 (e.g., parameters) between nodes in the set of nodes. In an example, the training set 515 is a subset of a complete training set. Here, the subset may enable processing nodes with limited storage resources to participate in training the ANN 505.

The training data may include multiple numerical values representative of a domain, such as red, green, and blue pixel values and intensity values for an image or pitch and volume values at discrete times for speech recognition. Each value of the training, or input 517 to be classified once ANN 505 is trained, is provided to a corresponding node 507 in the first layer or input layer of ANN 505. The values propagate through the layers and are changed by the objective function.

As noted above, the set of processing nodes is arranged to train the neural network to create a trained neural network. Once trained, data input into the ANN will produce valid classifications 520 (e.g., the input data 517 will be assigned into categories), for example. The training performed by the set of processing nodes 507 is iterative. In an example, each iteration of the training the neural network is performed independently between layers of the ANN 505. Thus, two distinct layers may be processed in parallel by different members of the set of processing nodes. In an example, different layers of the ANN 505 are trained on different hardware. The members of different members of the set of processing nodes may be located in different packages, housings, computers, cloud-based resources, etc. In an example, each iteration of the training is performed independently between nodes in the set of nodes. This example is an additional parallelization whereby individual nodes 507 (e.g., neurons) are trained independently. In an example, the nodes are trained on different hardware.

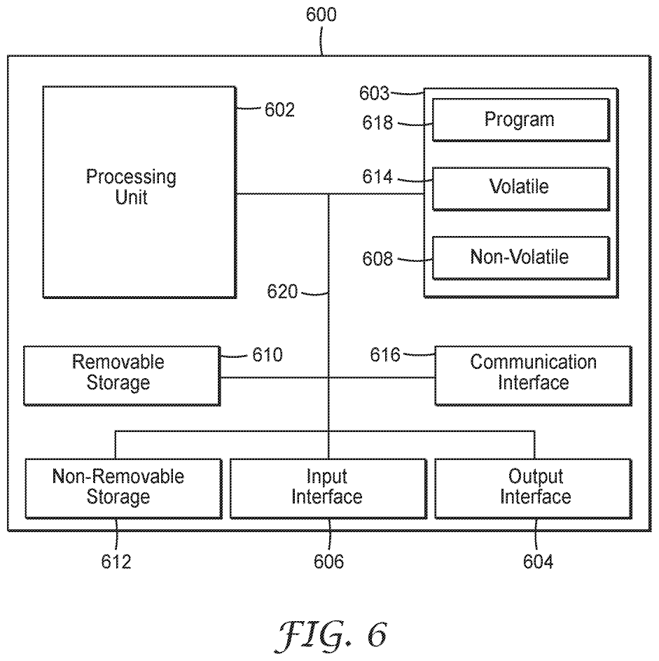

FIG. 6 is a block schematic diagram of a computer system 600 to implement code prediction process components and for performing methods and algorithms according to example embodiments. All components need not be used in various embodiments.

One example computing device in the form of a computer 600 may include a processing unit 602, memory 603, removable storage 610, and non-removable storage 612. Although the example computing device is illustrated and described as computer 600, the computing device may be in different forms in different embodiments. For example, the computing device may instead be a smartphone, a tablet, smartwatch, smart storage device (SSD), or other computing device including the same or similar elements as illustrated and described with regard to FIG. 6. Devices, such as smartphones, tablets, and smartwatches, are generally collectively referred to as mobile devices or user equipment.

Although the various data storage elements are illustrated as part of the computer 600, the storage may also or alternatively include cloud-based storage accessible via a network, such as the Internet or server based storage. Note also that an SSD may include a processor on which the parser may be run, allowing transfer of parsed, filtered data through I/O channels between the SSD and main memory.

Memory 603 may include volatile memory 614 and non-volatile memory 608. Computer 600 may include—or have access to a computing environment that includes—a variety of computer-readable media, such as volatile memory 614 and non-volatile memory 608, removable storage 610 and non-removable storage 612. Computer storage includes random access memory (RAM), read only memory (ROM), erasable programmable read-only memory (EPROM) or electrically erasable programmable read-only memory (EEPROM), flash memory or other memory technologies, compact disc read-only memory (CD ROM), Digital Versatile Disks (DVD) or other optical disk storage, magnetic cassettes, magnetic tape, magnetic disk storage or other magnetic storage devices, or any other medium capable of storing computer-readable instructions.

Computer 600 may include or have access to a computing environment that includes input interface 606, output interface 604, and a communication interface 616. Output interface 604 may include a display device, such as a touchscreen, that also may serve as an input device. The input interface 606 may include one or more of a touchscreen, touchpad, mouse, keyboard, camera, one or more device-specific buttons, one or more sensors integrated within or coupled via wired or wireless data connections to the computer 600, and other input devices. The computer may operate in a networked environment using a communication connection to connect to one or more remote computers, such as database servers. The remote computer may include a personal computer (PC), server, router, network PC, a peer device or other common data flow network switch, or the like. The communication connection may include a Local Area Network (LAN), a Wide Area Network (WAN), cellular, Wi-Fi, Bluetooth, or other networks. According to one embodiment, the various components of computer 600 are connected with a system bus 620.

Computer-readable instructions stored on a computer-readable medium are executable by the processing unit 602 of the computer 600, such as a program 618. The program 618 in some embodiments comprises software to implement one or more of the machine learning, converters, extractors, natural language processing machine, and other devices for implementing methods described herein. A hard drive, CD-ROM, and RAM are some examples of articles including a non-transitory computer-readable medium such as a storage device. The terms computer-readable medium and storage device do not include carrier waves to the extent carrier waves are deemed too transitory. Storage can also include networked storage, such as a storage area network (SAN). Computer program 618 along with the workspace manager 622 may be used to cause processing unit 602 to perform one or more methods or algorithms described herein.

### Examples

1. A computer implemented method includes receiving text-based clinical documentation corresponding to a patient treated at a healthcare facility, converting the text-based clinical documentation to create a machine compatible converted input having multiple features, providing the converted input to a trained machine learning model that has been trained based on a training set of historical converted clinical documentation by the first entity, and receiving a prediction from the trained machine learning model, wherein the prediction corresponds to at least one of a predicted diagnostic related group (DRG) code or a set of predictions comprising a predicted principal diagnosis code for provision to a DRG calculator to determine the DRG code.

2. The method of example 1 wherein converting the text-based clinical documentation comprises separating punctuation marks from text in the request and treating individual entities as tokens.

3. The method of example 2 wherein converting is performed by a natural language processing machine.

4. The method of any of examples 1-3 wherein set of predictions comprises one or more predicted secondary diagnosis codes and zero or more predicted procedure codes.

5. The method of any of examples 1-4 wherein the training set includes patient demographics from a patient information database.

6. The method of any of examples 1-5 wherein the machine learning model for predicting the DRG code is trained on the training set that includes an associated DRG code corresponding to each treated patient in the historical converted clinical documentation.

7. The method of any of examples 1-6 wherein the machine learning model for predicting the set of predictions is trained on the training set that includes an associated diagnosis or procedure code corresponding to each treated patient in the historical converted clinical documentation.

8. The method of example 7 wherein the training set includes multiple secondary diagnosis codes and procedure codes for one or more treated patients in the historical converted clinical documentation.

9. The method of any of examples 1-8 wherein the trained machine learning model comprises a classification model.

10. The method of any of examples 1-9 wherein the trained machine learning model comprises a recurrent or convolutional neural network.

11. A machine-readable storage device has instructions for execution by a processor of a machine to cause the processor to perform operations to perform a method. The operations include receiving text-based clinical documentation corresponding to a patient treated at a healthcare facility, converting the text-based clinical documentation to create a machine compatible converted input having multiple features, providing the converted input to a trained machine learning model that has been trained based on a training set of historical converted clinical documentation by the first entity, and receiving a prediction from the trained machine learning model, wherein the prediction corresponds to at least one of a predicted diagnostic related group (DRG) code or a set of predictions comprising a predicted principal diagnosis code for provision to a DRG calculator to determine the DRG code.

12. The device of example 11 wherein converting is performed by a natural language processing machine.

13. The device of any of examples 11-12 wherein the training set includes patient demographics from a patient information database.

14. The device of any of examples 11-13 wherein the machine learning model for predicting the DRG code is trained on the training set that includes an associated DRG code corresponding to each treated patient in the historical converted clinical documentation.

15. The device of any of examples 11-14 wherein the machine learning model for predicting the set of predictions is trained on the training set that includes an associated diagnosis or procedure code corresponding to each treated patient in the historical converted clinical documentation.

16. The device of example 15 wherein the training set includes multiple secondary diagnosis codes and procedure codes for one or more treated patients in the historical converted clinical documentation.

17. A device includes a processor and a memory device coupled to the processor and having a program stored thereon for execution by the processor to perform operation to perform a method. The operations include receiving text-based clinical documentation corresponding to a patient treated at a healthcare facility, converting the text-based clinical documentation to create a machine compatible converted input having multiple features, providing the converted input to a trained machine learning model that has been trained based on a training set of historical converted clinical documentation by the first entity, and receiving a prediction from the trained machine learning model, wherein the prediction corresponds to at least one of a predicted diagnostic related group (DRG) code or a set of predictions comprising a predicted principal diagnosis code for provision to a DRG calculator to determine the DRG code.

18. The device of example 17 wherein converting is performed by a natural language processing machine and wherein the training set includes patient demographics from a patient information database.

19. The device of any of examples 17-18 wherein the machine learning model for predicting the DRG code is trained on the training set that includes an associated DRG code corresponding to each treated patient in the historical converted clinical documentation.

20. The device of any of examples 17-19 wherein the machine learning model for predicting the set of predictions is trained on the training set that includes an associated diagnosis or procedure code corresponding to each treated patient in the historical converted clinical documentation and wherein the training set includes multiple secondary diagnosis codes and procedure codes for one or more treated patients in the historical converted clinical documentation.

Although a few embodiments have been described in detail above, other modifications are possible. For example, the logic flows depicted in the figures do not require the particular order shown, or sequential order, to achieve desirable results. Other steps may be provided, or steps may be eliminated, from the described flows, and other components may be added to, or removed from, the described systems. Other embodiments may be within the scope of the following claims.

1. A computer implemented method comprising:
receiving text-based clinical documentation corresponding to a patient treated at a healthcare facility;
converting the text-based clinical documentation to create a machine compatible converted input having multiple features;
providing the converted input to a trained machine learning model that has been trained based on a training set of historical converted clinical documentation by the first entity; and
receiving a prediction from the trained machine learning model, wherein the prediction corresponds to at least one of a predicted diagnostic related group (DRG) code or a set of predictions comprising a predicted principal diagnosis code for provision to a DRG calculator to determine the DRG code.

2. The method of claim 1 wherein converting the text-based clinical documentation comprises separating punctuation marks from text in the request and treating individual entities as tokens.

3. The method of claim 2 wherein converting is performed by a natural language processing machine.

4. The method of claim 1 wherein set of predictions comprises one or more predicted secondary diagnosis codes and zero or more predicted procedure codes.

5. The method of claim 1 wherein the training set includes patient demographics from a patient information database.

6. The method of claim 1 wherein the machine learning model for predicting the DRG code is trained on the training set that includes an associated DRG code corresponding to each treated patient in the historical converted clinical documentation.

7. The method of claim 1 wherein the machine learning model for predicting the set of predictions is trained on the training set that includes an associated diagnosis or procedure code corresponding to each treated patient in the historical converted clinical documentation.

8. The method of claim 7 wherein the training set includes multiple secondary diagnosis codes and procedure codes for one or more treated patients in the historical converted clinical documentation.

9. The method of claim 1 wherein the trained machine learning model comprises a classification model.

10. The method of claim 1 wherein the trained machine learning model comprises a recurrent or convolutional neural network.

11. A machine-readable storage device having instructions for execution by a processor of a machine to cause the processor to perform operations to perform a method, the operations comprising:
receiving text-based clinical documentation corresponding to a patient treated at a healthcare facility;
converting the text-based clinical documentation to create a machine compatible converted input having multiple features;
providing the converted input to a trained machine learning model that has been trained based on a training set of historical converted clinical documentation by the first entity; and
receiving a prediction from the trained machine learning model, wherein the prediction corresponds to at least one of a predicted diagnostic related group (DRG) code or a set of predictions comprising a predicted principal diagnosis code for provision to a DRG calculator to determine the DRG code.

12. The device of claim 11 wherein converting is performed by a natural language processing machine.

13. The device of claim 11 wherein the training set includes patient demographics from a patient information database.

14. The device of claim 11 wherein the machine learning model for predicting the DRG code is trained on the training set that includes an associated DRG code corresponding to each treated patient in the historical converted clinical documentation.

15. The device of claim 11 wherein the machine learning model for predicting the set of predictions is trained on the training set that includes an associated diagnosis or procedure code corresponding to each treated patient in the historical converted clinical documentation.

16. The device of claim 15 wherein the training set includes multiple secondary diagnosis codes and procedure codes for one or more treated patients in the historical converted clinical documentation.

17. A device comprising:
a processor; and
a memory device coupled to the processor and having a program stored thereon for execution by the processor to perform operation to perform a method, the operations comprising:
receiving text-based clinical documentation corresponding to a patient treated at a healthcare facility;
converting the text-based clinical documentation to create a machine compatible converted input having multiple features;
providing the converted input to a trained machine learning model that has been trained based on a training set of historical converted clinical documentation by the first entity; and
receiving a prediction from the trained machine learning model, wherein the prediction corresponds to at least one of a predicted diagnostic related group (DRG) code or a set of predictions comprising a predicted principal diagnosis code for provision to a DRG calculator to determine the DRG code.

18. The device of claim 17 wherein converting is performed by a natural language processing machine and wherein the training set includes patient demographics from a patient information database.

19. The device of claim 17 wherein the machine learning model for predicting the DRG code is trained on the training set that includes an associated DRG code corresponding to each treated patient in the historical converted clinical documentation.

20. The device of claim 17 wherein the machine learning model for predicting the set of predictions is trained on the training set that includes an associated diagnosis or procedure code corresponding to each treated patient in the historical converted clinical documentation and wherein the training set includes multiple secondary diagnosis codes and procedure codes for one or more treated patients in the historical converted clinical documentation.
